(19) United States
(12) Patent Application Publication
(10) Pub. No.: US 2004/0259184 A1
(43) Pub. Date: Dec. 23, 2004

(54) Fluorescent methods for the high-throughput quantification of tartrate-resistant acid phosphatase (TRAP)

(75) Inventors: Lee Albee II, St. Louis, MO (US); Gabriel Mbalaviele, O'Fallon, MO (US)

Correspondence Address: PHARMACIA CORPORATION, GLOBAL PATENT DEPARTMENT, POST OFFICE BOX 1027, ST. LOUIS, MO, 63006, US

(73) Assignee: Pharmacia Corporation

(21) Appl. No.: 10/840,818

(22) Filed: May 7, 2004

Related U.S. Application Data
(60) Provisional application No. 60/468,593, filed on May 7, 2003.

(51) Int. Cl. C12Q 1/42

(52) U.S. Cl. 435/21; 435/40.5

(57) ABSTRACT

Methods for quantifying tartrate-resistant acid phosphotase (TRAP) activity, or determining the distribution of TRAP in a tissue are provided. More specifically, the fluorescent assay of the present invention is useful for identifying or quantifying TRAP in isolated osteoclast cells or osteoclasts differentiated from RAW264.7 monocyte-macrophage cells.

This application claims priority to U.S. Application Ser. No. 60/468,593 filed on May 7, 2003.

### FIELD OF THE INVENTION

The following invention is directed to methods for quantifying tartrate-resistant acid phosphotase (TRAP) activity. More specifically, the fluorescent assay of the present invention is useful for identifying or quantifying TRAP in isolated osteoclast cells or osteoclasts differentiated from RAW264.7 monocyte-macrophage cells.

### BACKGROUND OF THE INVENTION

Bone remodeling is a continuous balance between bone formation and resorption. This cooperative balance in bone metabolism is a stringently regulated process with the purpose of maintaining bone homeostasis. In physiological bone remodeling, bone formation is regulated by osteoblasts while bone resporption is controlled by osteoclasts. Deviation from the normal bone remodeling balance can result in bone diseases such as osteoporosis, osteopetrosis, and periodontal disease.

Several model systems are commonly used to study osteoclast differentiation and activity. For example, the Raw 264.7 cell line is used to study markers of osteoclast differentiation. Raw 264.7 cells can be differentiated into osteoclast-like cells (OCL cells) via the addition of RANKL (receptor activator of nuclear factor kappa B ligand), also referred to as ODF (osteoclast differentiation factor), and M-CSF (marcrophage-colony stimulating factor) to the growth media. The OCL cells are capable of expressing various biochemical markers of osteoclast differentiation. One such marker is tartrate-resistant acid phophotase.

Tartrate-resistant acid phosphotase (TRAP) is an ion-containing enzyme selectively expressed in bone-resorbing osteoclasts. TRAP has been proposed as a serum/plasma marker for osteoclast activity because it is elevated in the serum or plasma of patient groups known to have increased bone metabolism. The TRAP enzyme is thought to have a role in bone resorption, because knock-out TRAP mice have mild osteoporosis. Further, it is known that TRAP is secreted into the circulation during bone resorption.

TRAP is used as a standard histochemical marker of osteoclasts harvested from bone or cells differentiated into osteoclast-like cells (OCL). For example, Lau et al. (Clin. Chem. 343: 458-462, 1987) describes a spectrophotometric assay of TRAP. Even so, existing methods of quantifying TRAP positive cells involve manual scoring of colorimetric dye changes, and are not suitable for high-throughput screening protocols needed in drug compound screening or target discovery/ validation.

Other TRAP assays involve immunohistochemistry. For example, Cheung et al. (Clin. Chem. 41: 679-686, 1995) describe an ELISA developed from rabbit TRAP polyclonal antibodies, and Helleen et al. (J. Bone Miner. Res. 13(4): 683-687, 1998) describe a fluorescent, direct two-site immunoassay for TRAP. However, immunoassays are inefficient for cell-based formats.

### SUMMARY OF THE INVENTION

As evidenced by the continuing research into the differentiation and activity of osteoclasts, and by the shortcomings of the processes of the art, there still remains a need for high-throughput assays to quantify TRAP activity. Such methods would provide more efficient, cost-effective, or faster means of quantifying TRAP activity and osteoclast numbers.

Methods are provided for providing a fluorescent assay for high-throughput quantification of tartrate-resistant acid phosphatase activity.

The assay of the present invention is useful, for example, in identifying or quantifying TRAP in osteoclasts differentiated from RAW264.7 monocyte-macrophage cells or biological fluid samples or biological extracts, identifying osteoclast modulating drugs or agents, screening gene libraries for their phenotypic effects on osteoclastogenesis, diagnosing metabolic bone disorders such as osteoporosis or postmenopausal rapid bone loss, monitoring the efficacy of therapeutic regimens designed to treat such disorders, determining the extent of imbalances between bone formation and resorption, or detecting bone destruction from Paget's disease or malignancies.

### DESCRIPTION OF THE DRAWINGS

FIG. 1 is a series of photographic images illustrating a time-course of differentiating RAW cells stained with PI (red) and stained for TRAP activity (yellow) to demonstrate the formation of OCL over the course of six days.

FIG. 2 is a graph depicting the increased TRAP fluorescence detected during the differentiation of RAW cells over time into OCL.

FIG. 3 is two graphs depicting the results of the automated counting of size selected cells as well as the count of TRAP positive cells.

FIG. 4 is a graph depicting the amount of overall TRAP fluorescence detected at day 6 in the presence or absence of MAPK inhibitors SB 203580 and 202190 (p38 MAPK inhibitory compounds), SB202190 (an inactive analog of SB 202190), and PD98059 (MEK1 inhibitor).

FIG. 5 is a graph depicting the quantity of TRAP positive cells counted at day 6 in the presence or absence of MAPK inhibitors SB 203580 and 202190 (p38 MAPK inhibitory compounds), SB202190 (an inactive analog of SB 202190), and PD98059 (MEK1 inhibitor).

### DETAILED DESCRIPTION OF THE INVENTION

Disclosed is a high-throughput fluorescent assay to quantitatively measure tartrate-resistant acid phosphatase (TRAP) activity and osteoclast numbers.

One embodiment of the invention is directed to an assay device comprising incubating a reaction mixture comprising cells, fluorescently-labeled phosphatase substrate, buffer and tartrate; washing the mixture, and determining the quantity of TRACE present.

Another embodiment of the invention provides a method for identifying the level of activity of the TRAP enzyme in isolated osteoclast cells.

In another embodiment, the present invention is directed to a fluorescent assay for the high throughput quantification of TRAP (tartrate-resistant acid phosphatase) activity within osteoclasts differentiated from RAW264.7 monocyte-macrophage cells.

In another embodiment of the present invention, the efficacy of a osteoclast modulating drug or agent is assessed by quantifying the TRACE content in samples obtained before and after administration of the osteoclast modulating drug or agent.

In another embodiment of the present invention, the assay is used for screening gene libraries for their phenotypic effects on osteoclastogenesis.

In another embodiment, the present invention comprises a method of identifying and quantifying TRAP activity in a biological fluid sample or biological extract.

In another embodiment of the present invention comprises a method of screening for the level of bone resorption in a mammalian subject.

In another embodiment, the present invention comprises a method of detecting the presence or absence of osteoclasts in a biological extract.

In another embodiment, the present invention comprises diagnosing metabolic bone disorders such as osteoporosis or postmenopausal rapid bone loss in a mammal.

In another embodiment, the present invention comprises monitoring a change in the status of a bone resorption condition in a mammal.

In another embodiment, the present invention comprises monitoring the efficacy of therapeutic regimens designed to treat bone resporption disorders.

In another embodiment, the present invention comprises a kit containing a high-throughput fluorescent assay to quantify TRAP activity.

These detailed descriptions are presented for illustrative purposes only and are not intended to be, and should not be taken as, a restriction to the scope of the invention or the claims that follow. Rather, they are merely some of the embodiments that one skilled in the art would understand from the entire contents of this disclosure.

All parts are by weight and temperatures are in Degrees centigrade unless otherwise indicated.

I. Abbreviations and Definitions

The following is a list of abbreviations and the corresponding meanings as used interchangeably herein:

The term mg means milligram

The term ml or mL means milliliter

The term μg or ug means microgram

The term μl or ul means microliter

The term CV means coefficient of variance

The term DMEM means Dulbecco's Modified Eagle Medium

The term DMSO means Dimethyl Sulfoxide

The term ELF means enzyme-labeled fluorescence

The term FBS means fetal bovine serum

The term MAPK means mitogen-activated protein kinase

The term M-CSF means macrophage-colony stimulating factor

The term MEK1 means mitogen extracellular kinase-1

The term MEM means Minimum Essential Medium

The term OCL means osteoclast like cells

The term ODF means osteoclast differentiation factor

The term PBS means Phosphate Buffer Saline

The term PI means propidium iodide

The term RANKL means receptor activator of nuclear factor kappa B ligand

The term TRAP means tartrate-resistant acid phosphatase

As used herein, the term “osteoclast modulating agent” means a compound, composition of matter, pharmaceutical, chemical, or any other treatment and combinations thereof, administered for the purpose of therapeutically increasing or decreasing osteoclast migration, adhesion, or activity. Osteoclast modulating agents include both naturally occurring and synthetic drugs and agents. A non-limitative list of osteoclast modulating drugs or agents includes estrogen, estrogen receptor modulators (such as SERMs), osteoprotegerin (OPG) inhibitors (such as osteoclast differentiation and activation receptor), bone morphogenic factors (such as BMP-1 to BMP-12), TGF-β and TGF-β family members, fibroblast growth factors (such as FGF-1 to FGF-10), interleukin-1 (IL-1) inhibitors, TNF-α inhibitors, αᵥβ₃ or dual αᵥβ₃/αᵥβ₅ inhibitors, parathyroid hormone, E series prostaglandins, bisphoshponates, or bone-enhancing minerals such as fluoride or calcium.

The term “biological extract” means any body fluid or tissue containing cells which may be tested to determine the presence or absence of TRAP, including, but not limited to blood or a blood component, saliva, urine, or bone tissue. The sample may come from human patients, or from other mammals, or from birds.

The term “Bone resorption abnormality” or “bone resorption condition” refers to a condition characterized by an elevated level of bone degradation (resorption) in a mammalian subject. Bone resorption conditions include osteopetrosis, osteoporosis, osteomyelitis, hypercalcemia, osteonecrosis, bone loss due to rheumatoid arthritis, periodontal bone loss, immobilization, or prosthetic loosing, Paget's disease, bone cancers (e.g., metastases in bone), osteomalacia, rickets, renal osteodystrophy, and osteopenia brought on by surgery or steroid administration.

II. Assay Method

In one embodiment, the present invention is directed to an assay for detecting tartrate resistant acid phosphatase (TRAP) activity, comprising

incubating a reaction mixture comprising cells, fluorescently-labeled phosphatase substrate, buffer and tartrate;

allowing phosphatase-mediated cleavage of the phosphatase substrate;

determining the amount of TRAP activity from the fluorescence intensity of the cleaved substrate.

Assays of the present invention are useful for determining the activity of TRAP in or on osteoclast or osteoclast-like cells. Osteoclast cells are formed via cell-cell fusion after contact with hematopoietic precursors. For example, Morishima et al. describe three different methods of osteoclast differentiation, namely mouse bone marrow cell culture, co-culture of mouse spleen cells with stromal cells, and RAW264.7 cell cultures (J Endocrinol 2003 February;176(2):285-92). In one embodiment, cells to be assayed are osteoclast-like cells (OCL cells) derived from RAW 264.7 monocyte-macrophage cells via the addition of receptor activator of nuclear factor kappa B ligand (RANKL) and macrophage-colony stimulating factor (M-CSF) (Example 1). Other osteoclast precursor cells lines are known to those skilled in the art. For instance, other osteoclast precursor cell lines include but are not limited to MOCP-5, peripheral blood mononuclear precursors such as Cd34 selected primary monocytes, synovial cells, or the generation of osteoclastogenic cell lines immortalized with SV-40 large T antigen (J. Bone Miner Res. 13(7):1112-1123).

In a further embodiment, the cells are osteoclast cell lines. In another embodiment, the cells can be osteoclasts isolated from bone.

In yet another embodiment, cells are contained in biological fluid samples or biological extracts. Assays of the present invention are also useful for determining the quantity of osteoclast cells in a biological fluid sample or biological extracts. Such biological fluid samples or biological extracts may further be treated with hematopoietic precursors to induce osteoclast differentiation.

The assay of the present invention contemplates, for instance, an in situ format. (Example 2). Cells can be fixed to a plated surface using a variety of techniques known to those skilled in the art. For instance, the cells can be fixed to a plate with a chemical reagent. The chemical reagent can be any of the reagents commonly used for the purpose of fixing cells or tissue sections. Examples of the chemical reagents include, but are not limited to, formaldehyde, paraformaldehyde, formatin, methanol, and the like. Further, cells of the present invention can be permeablized by methods well known to those skilled in the art. In one embodiment, cells may be permeablized in PBS. In another embodiment, the PBS contains Tween® (Sigma, St. Louis, Mo.).

The assay of the present invention provides a fluorescently-labeled phosphatase substrate (Example 2). In a preferred embodiment, the substrate for assaying phosphatase activity is the fluorogenic ELF-97® phosphatase substrate (ELF-97® phosphatase, Molecular Probes, Eugene, Oreg.). ELF-97® phosphatase is expected to yield a photostable yellow-green fluorescent precipitate after the substrate is converted to precipitate. In another embodiment, the labeled phosphatase substrate is a chromogenic indolyl substrate (Biotium, Inc. Hayward, Calif.). The indolyl phosphatases (B8406®, B-8410®) produce dark blue and magenta precipitates, respectively. In another embodiment, the fluorescent label is Fast Red Violet LB® (FRV) (SIGMA Chemical, St. Louis, Mo.). In yet another embodiment, the fluorescent label is AttoPhos® System (Promega, Madison, Wis.). Other fluorescent labels will occur to those skilled in the art, and these are within the intended scope of the invention.

The tartrate-resistant acid phosphatase activity measured by the present invention can be distinguished from other phosphatase enzymes' activity when carried out under the conditions of slightly acidic pH and high concentrations of tartrate. In one embodiment, the buffered solution of the present invention is from about pH 4.85 to about pH 5.15. In a preferred embodiment, the pH of the buffer is about 5.0. In another embodiment, the buffer is selected from itrate, malonate, lactate, trichloroacetate, sulfosalicylate, tartarate, phosphates, borates, acetates, piperazine-N,N′-bis(2-hydroxypropane)sulfonic acid (POPSO), N-2-hydroxythylpiperazine-N′-2-ethanesulfonic acid (H EP ES), 3-N-(trishydroxymethyl)methylamino-2-hydroxypropanesulfonic acid (TAPSO), and 2-([atris-(hydroxymethyl)methyl]amino)ethanesulfonic acid (TES); or combinations thereof. In a preferred embodiment, the buffer is an acetate.

In one embodiment of the present invention, the concentration of tartrate is from about 6.0 mM to about 7.4 mM. Preferably, the concentration tartrate is about 6.7 mM. In another embodiment, the assay utilizes a source of tartrate. Sources of tartrate include, for example, tartrate salts, such as copper, sodium or potassium tartrate.

Incubated cells can then be washed in, for example, PBS, preferably from about 1 to about 5 times. In another embodiment, the plated cells are washed from about 2 to about 4 times.

Quantification of TRAP activity is measured according to fluorescence intensity (Example 3). In one embodiment, an energy source is used to emit an energy beam sufficient to induce fluorescence in the cleaved fluorescent substrate. In another embodiment, the measurements are be taken by a detector for detecting light emitted by the cleaved substrate. In yet another embodiment, the fluorescent activity of the cleaved substrate is measurably altered in relation to the concentration of the fluorescent phosphatase substrate. A detector for detecting fluorescence emitted by the cleaved substrate is also contemplated by the assay of the present invention. In another embodiment, the detector measures the difference in fluorescence intensity between the reaction mixture and the cleaved substrate.

An assay for detecting the presence of tartrate resistant acid phosphotase (TRAP) is further contemplated by the present invention (Example 4). In one embodiment, TRAP is detected by incubating a reaction mixture comprising cells; fluorescently-labeled phosphatase substrate; an acidic buffer; and tartrate; washing the mixture; and determining the quantity of TRAP in the cells from the intensity of fluorescence.

Further, quantification of osteoclast cells can be extrapolated from the presence of TRAP (Example 5). In one embodiment, TRAP activity is used to determine osteoclast number or the quantification of differentiation into OCL. In a preferred embodiment, TRAP staining intensity is used. In another embodiment, cell size in addition to TRAP activity is used to determine osteoclast number or OCL differentiation. In yet another embodiment, mononucleation is used to quantify osteoclast or OCL number. In another embodiment, the quantification of OCL cells is contemplated by the assay of the present invention. A time course of quantification of osteoclast cells is also contemplated by the present invention.

The assays and techniques described above may be used advantageously to screen rapidly large numbers of osteoclast modulating drugs or agents (Examples 6 and 7). The assays and techniques of the present invention may be automated to screen compounds generated in phage display, synthetic peptide and chemical synthesis libraries. In another embodiment, the assay of the present invention is amenable for medium to high throughput analysis of compound libraries for impact upon OCL formation.

The assays and techniques of the present invention may further be used to screen gene libraries for genes that effect osteoclastagenesis.

In one embodiment, the format is multiwell plates, preferably from about 6 wells to about 384 wells. It is preferred that the assay is conducted on black-well plates, but clear coated or other colored plates are also contemplated.

III. Methods of Use

In one embodiment, the present invention provides a method of screening for or monitoring the level of bone resorption in a mammalian subject, comprising:

obtaining a biological fluid sample or biological extract from a mammalian subject, and determining a level of TRAP in the sample wherein a determined level which is above that characteristic of normal subjects is an indication that the subject has a bone resorption abnormality. In another embodiment, the bone resorption abnormality is osteoporosis. In another embodiment, the condition may be Paget's disease, and conditions related to the progress of benign and malignant tumors of the bone and metastatic cancers that have migrated to bone cells from elsewhere in the body, e.g., from prostate or breast initial tumors. Other conditions associated with changes in bone metabolism include osteomalacial diseases, rickets, abnormal growth in children, renal osteodystrophy, and drug-induced osteopenia. Further, abnormalities in bone metabolism are often side effects of thyroid treatment and thyroid conditions per se, such as primary hypothyroidism and thyrotoxicosis as well as Cushing's disease.

In yet another embodiment, the present invention provides a method of monitoring a change in the status of a bone resorption condition in a subject, in response to a therapeutic treatment, which further includes measuring the level of said TRAP in a biological extract from the subject during or after such treatment.

The method may also be used in veterinary applications for detecting and monitoring bone resorption abnormalities that occur among farm animals and house pets. Conditions that produce destruction of alveolar bone are common among cats, horses and dogs, and often require veterinary treatment. The method may also be used with other diagnostic methods, such as radiographic techniques and assays directed to other indicators of bone resorption status, to provide a fuller picture of the subject's status.

In another embodiment of the present invention, the efficacy of a osteoclast modulating drug or agent candidate is assessed by quantifying the TRACE content in samples obtained before and after administration of the osteoclast modulating drug or agent candidate. A change in TRAP content or activity from an abnormal level towards a more normal level is expected if the osteoclast modulating drug or agent is effective. This approach can be used both experimentally in the development of new osteoclast modulating drugs or agents, or in connection with therapy to determine if the administration of a therapeutic agent such as a growth modulating drug or agent is effective in the treatment of a osteoclast disorder. In another embodiment, multiple osteoclast modulating drug or agent candidates can be screened simultaneously in a multi-well format.

In another embodiment, the present invention contemplates a method of evaluating an osteoclast modulating drug or agent candidate comprising:

(a) adding the candidate to a composition comprising cells, fluorescently-labeled phosphatase substrate, buffer and tartrate;

(b) washing said composition; and

(c) determining the level of activity of the reaction, wherein a change in the level between the presence and absence of the candidate indicates that the candidate is a osteoclast modulating drug or agent.

In another embodiment, the present invention contemplates TRAP modulating compounds useful for treating or preventing bone resorption. A TRAP modulator of the present invention can be administered to the subject as the neat compound alone. Alternatively the TRAP modulator of the present invention can be presented with one or more pharmaceutically acceptable excipients in the form of a pharmaceutical composition. A useful excipient can be, for example, a carrier. The carrier must, of course, be acceptable in the sense of being compatible with the other ingredients of the composition and must not be deleterious to the recipient. The carrier can be a solid or a liquid, or both, and is preferably formulated with the compound as a unit-dose composition, for example, a tablet, which can contain from 0.05% to 95% by weight of the active compound. Other pharmacologically active substances can also be present, including other compounds of the present invention. The pharmaceutical compositions of the invention can be prepared by any of the well known techniques of pharmacy, consisting essentially of admixing the components.

These compounds can be administered by any conventional means available for use in conjunction with pharmaceuticals, either as individual therapeutic compounds or as a combination of therapeutic compounds.

The amount of TRAP modulator which is required to achieve the desired biological effect will, of course, depend on a number of factors such as the specific compound chosen, the use for which it is intended, the mode of administration, and the clinical condition of the recipient.

In general, a daily dose can be in the range of from about 0.01 to about 100 mg/kg bodyweight/day, preferably from about 0.05 mg to about 50 mg/kg bodyweight/day, more preferably from about 0.01 to about 20 mg/kg bodyweight/day. Even more preferably from about 0.01 to about 10 mg/kg bodyweight/day. This total daily dose can be administered to the patient in a single dose, or in proportionate multiple subdoses. Subdoses can be administered 2 to 6 times per day. Doses can be in sustained release form effective to obtain desired results.

Orally administrable unit dose formulations, such as tablets or capsules, can contain, for example, from about 0.1 to about 1000 mg of the compound, preferably about 1 to about 500 mg of compound, more preferably from about 2 to about 400 mg of compound, even more preferably from about 2 to about 200 mg of compound, further preferably from about 2 to about 100 mg of compound, even further preferably from about 2 to about 50 mg of compound. In the case of pharmaceutically acceptable salts, the weights indicated above refer to the weight of the ion derived from the salt.

Oral delivery of the TRAP modulator of the present invention can include formulations, as are well known in the art, to provide prolonged or sustained delivery of the drug to the gastrointestinal tract by any number of mechanisms. These include, but are not limited to, pH sensitive release from the dosage form based on the changing pH of the small intestine, slow erosion of a tablet or capsule, retention in the stomach based on the physical properties of the formulation, bioadhesion of the dosage form to the mucosal lining of the intestinal tract, or enzymatic release of the active drug from the dosage form. The intended effect is to extend the time period over which the active drug molecule is delivered to the site of action by manipulation of the dosage form. Thus, enteric-coated and enteric-coated controlled release formulations are within the scope of the present invention. Suitable enteric coatings include cellulose acetate phthalate, polyvinylacetate phthalate, hydroxypropylmethylcellulose phthalate and anionic polymers of methacrylic acid and methacrylic acid methyl ester.

When administered intravenously, the daily dose can, for example, be in the range of from about 0.1 mg/kg body weight to about 20 mg/kg body weight, preferably from about 0.25 mg/kg body weight to about 10 mg/kg body weight, more preferably from about 0.4 mg/kg body weight to about 5 mg/kg body weight. This dose can be conveniently administered as an infusion of from about 10 ng/kg body weight to about 2000 ng/kg body weight per minute. Infusion fluids suitable for this purpose can contain, for example, from about 0.1 ng to about 10 mg, preferably from about 1 ng to about 200 mg per milliliter. Unit doses can contain, for example, from about 1 mg to about 200 g of the compound of the present invention. Thus, ampoules for injection can contain, for example, from about 1 mg to about 200 mg.

Pharmaceutical compositions according to the present invention include those suitable for oral, rectal, topical, buccal (e.g., sublingual), and parenteral (e.g., subcutaneous, intramuscular, intradermal, or intravenous) administration, although the most suitable route in any given case will depend on the nature and severity of the condition being treated and on the nature of the particular compound which is being used. In most cases, the preferred route of administration is oral.

Formulations suitable for topical administration to the eye also include eye drops wherein the active ingredients are dissolved or suspended in suitable carrier, especially an aqueous solvent for the active ingredients. For periodontal disease, a TRAP modulating formulation can be applied topically to the gum or tooth. The TRAP modulating ingredients are preferably present in such formulations in a concentration of 0.5 to 20%, advantageously 0.5 to 10% and particularly about 1.5% w/w.

Pharmaceutical compositions suitable for oral administration can be presented in discrete units, such as capsules, cachets, lozenges, or tablets, each containing a predetermined amount of at least one compound of the present invention; as a powder or granules; as a solution or a suspension in an aqueous or non-aqueous liquid; or as an oil-in-water or water-in-oil emulsion. As indicated, such compositions can be prepared by any suitable method of pharmacy which includes the step of bringing into association the active compound(s) and the carrier (which can constitute one or more accessory ingredients). In general, the compositions are prepared by uniformly and intimately admixing the active compound with a liquid or finely divided solid carrier, or both, and then, if necessary, shaping the product. For example, a tablet can be prepared by compressing or molding a powder or granules of the compound, optionally with one or more assessory ingredients. Compressed tablets can be prepared by compressing, in a suitable machine, the compound in a free-flowing form, such as a powder or granules optionally mixed with a binder, lubricant, inert diluent and/or surface active/dispersing agent(s). Molded tablets can be made by molding, in a suitable machine, the powdered compound moistened with an inert liquid diluent.

Pharmaceutical compositions suitable for buccal (sub-lingual) administration include lozenges comprising a compound of the present invention in a flavored base, usually sucrose, and acacia or tragacanth, and pastilles comprising the compound in an inert base such as gelatin and glycerin or sucrose and acacia.

Pharmaceutical compositions suitable for parenteral administration conveniently comprise sterile aqueous preparations of a compound of the present invention. These preparations are preferably administered intravenously, although administration can also be effected by means of subcutaneous, intramuscular, or intradermal injection. Such preparations can conveniently be prepared by admixing the compound with water and rendering the resulting solution sterile and isotonic with the blood. Injectable compositions according to the invention will generally contain from 0.1 to 5% w/w of a compound disclosed herein.

Pharmaceutical compositions suitable for rectal administration are preferably presented as unit-dose suppositories. These can be prepared by admixing a compound of the present invention with one or more conventional solid carriers, for example, cocoa butter, and then shaping the resulting mixture.

Pharmaceutical compositions suitable for topical application to the skin preferably take the form of an ointment, cream, lotion, paste, gel, spray, aerosol, or oil. Carriers which can be used include vaseline, lanoline, polyethylene glycols, alcohols, and combinations of two or more thereof. The active compound is generally present at a concentration of from 0.1 to 15% w/w of the composition, for example, from 0.5 to 2%.

Transdermal administration is also possible. Pharmaceutical compositions suitable for transdermal administration can be presented as discrete patches adapted to remain in intimate contact with the epidermis of the recipient for a prolonged period of time. Such patches suitably contain a compound of the present invention in an optionally buffered, aqueous solution, dissolved and/or dispersed in an adhesive, or dispersed in a polymer. A suitable concentration of the active compound is about 1% to 35%, preferably about 3% to 15%. As one particular possibility, the compound can be delivered from the patch by electrotransport or iontophoresis.

In any case, the amount of active ingredient that can be combined with carrier materials to produce a single dosage form to be administered will vary depending upon the host treated and the particular mode of administration.

The solid dosage forms for oral administration including capsules, tablets, pills, powders, and granules noted above comprise one or more compounds of the present invention admixed with at least one inert diluent such as sucrose, lactose, or starch. Such dosage forms may also comprise, as in normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration can include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents.

Injectable preparations, for example, sterile injectable aqueous or oleaginous suspensions may be formulated according to the known art using suitable dispersing or setting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Pharmaceutically acceptable carriers encompass all the foregoing and the like.

Treatment Regimen

The dosage regimen to prevent, give relief from, or ameliorate a disease condition with TRAP modulators of the present invention is selected in accordance with a variety of factors. These include the type, age, weight, sex, diet, and medical condition of the patient, the severity of the disease, the route of administration, pharmacological considerations such as the activity, efficacy, pharmacokinetics and toxicology profiles of the particular compound employed, whether a drug delivery system is utilized, and whether the compound is administered as part of a drug combination. Thus, the dosage regimen actually employed may vary widely and therefore deviate from the preferred dosage regimen set forth above.

Initial treatment of a patient suffering from an osteoclast resorption disorder can begin with the dosages indicated above. Treatment should generally be continued as necessary over a period of several weeks to several months or years until the disease condition has been controlled or eliminated. Patients undergoing treatment with the compounds or compositions disclosed herein can be routinely monitored by, for example, measuring bone density by any of the methods well known in the art, to determine the effectiveness of therapy. Continuous analysis of such data permits modification of the treatment regimen during therapy so that optimal effective amounts of TRAP modulators of the present invention are administered at any point in time, and so that the duration of treatment can be determined as well. In this way, the treatment regimen/dosing schedule can be rationally modified over the course of therapy so that the lowest amount of the compound of the present invention which exhibits satisfactory effectiveness is administered, and so that administration is continued only so long as is necessary to successfully treat the condition.

Assays and techniques of the present invention can be used for screening gene libraries for their phenotypic effects on osteoclastogenesis. In one embodiment of the present invention, the effect of a gene can be assessed by quantifying the TRACE content or activity in samples obtained before and after addition of the gene. In another embodiment, multiple genes can be assayed simultaneously. In another embodiment, gene libraries can be screened.

Kits for measuring the levels of TRAP in patient samples are also within the scope of the present invention. Such assay kits would contain fluorescently-labeled phosphatase, preferably ELF-97® phosphatase substrate (ELF-97® phosphatase, Molecular Probes, Eugene, Oreg.).

IV. Examples

Having now generally described the invention, the same will be more readily understood through reference to the following examples which are provided by way of illustration, and are not intended to be limiting of the present invention, unless specified.

### EXAMPLE 1

Cell culture

RAW (RAW 264.7 monocyte-macrophage) RAW (ATTC, Manassas, Va.) cells were maintained in culture using DMEM (Dulbecco's Modified Eagle Medium) media containing L-glutamine at 584 mg/L, 1% penicillin/streptomycin, and 10% FBS (fetal bovine serum). For osteoclast differentiation RAW cells were put down onto 96 well plates at a density of 1,000 cells per well in alpha MEM (Minimum Essential Medium) media containing L-glutamine at 292 mg/L, 1% penicillin/streptomycin, 10% FBS, soluble RANKL (PeproTech, Rocky Hill, N.J.) at 10 ng/ml, and M-CSF (Cell Sciences, Norwood, Mass.) at 25 ng/ml. All media were changed every two days. For experiments with compounds, the compounds were added at the time of plating the cells in differentiation media. Additionally compounds were replenished during every media change leading up to the day 6 termination of the experiment.

### EXAMPLE 2

TRAP staining.

Media was removed and cells were fixed onto their plated surface with 4% formaldehyde (Sigma, Saint Louis, Mo.) in PBS (Phosphate Buffer Saline) solution for 20 minutes. After fixing, cells were permeablized in PBS with 0.1% Tween (Sigma, Saint Louis, Mo.) for 15 minutes. Cells were then incubated with 200 uM of ELF 97 phosphatase substrate (Molecular Probes, Eugene, Oreg.) for 15 minutes in a 3.4 mM acetate buffered solution (pH 5.0) containing 6.7 mM Tartrate (Sigma, Saint Louis, Mo.). These conditions (of slightly acidic pH and high concentrations of tartrate) discriminate TRAP activity from other phosphatases. Cells were then washed twice with PBS (pH 7.4). After the final wash, plates were immediately quantified for TRAP activity.

### EXAMPLE 3

Quantification of TRAP TRAP activity per well was quantified on a SpetraFluor Plus (Tecan, Research Triangle Park, N.C.) using an excitation of 360 nm and an emission of 550 nm using the optimal gain function. For quantification of the number of OCL, PI (propidium iodide) (Sigma, Saint Louis, Mo.) at a final concentration of 10 ug/ml was added to the TRAP stained cells to nonspecifically stain the cells. After PI staining the 96 well plates were analyzed using an ArrayScanII (Cellomics, Pittsburgh, Pa.) on the Nuclear Translocation software. In this Nuclear Translocation software analysis, the cytoplasmic image area is not used while the nuclear image area is expanded to encompass the entire PI stained cellular area. Cell size selection was performed through nuclear size selection criteria within Channel 1 of the Nuclear Translocation software package. TRAP intensity selection was performed through total fluorescence intensity utilizing Channel 2.

### EXAMPLE 4

Fluorescent TRAP staining of cells.

The fluorescent phosphatase substrate ELF-97[2-(5′-chloro-2-phosphoryloxyphenyl)-6-chloro-4 (3H)-quinazolinone] was used to stain, in situ, RAW cells for TRAP activity. Acid phosphatase mediated cleavage of the ELF-97 yields an extremely fine precipitate that remains localized to the site of enzymatic activity and fluoresces, with maximal excitation at approximately 360 nm and maximal emission at approximately 530 nm.

Data acquisition and analysis was handled by an automated fluorescent microscope and image analysis program which allows for the rapid quantification of TRAP positive cells. Data gathered in this system included: 1) number of TRAP positive cells compared to total number of cells present, 2) percentage of multinucleated cells which are TRAP positive, 3) percentage of TRAP positive cells that are multinucleated, and 4) the overall TRAP fluorescent intensity (average) measured across all TRAP positive cells. Additionally, this method can be extended to measure total TRAP activity, per well, utilizing a spectrafluorometer.

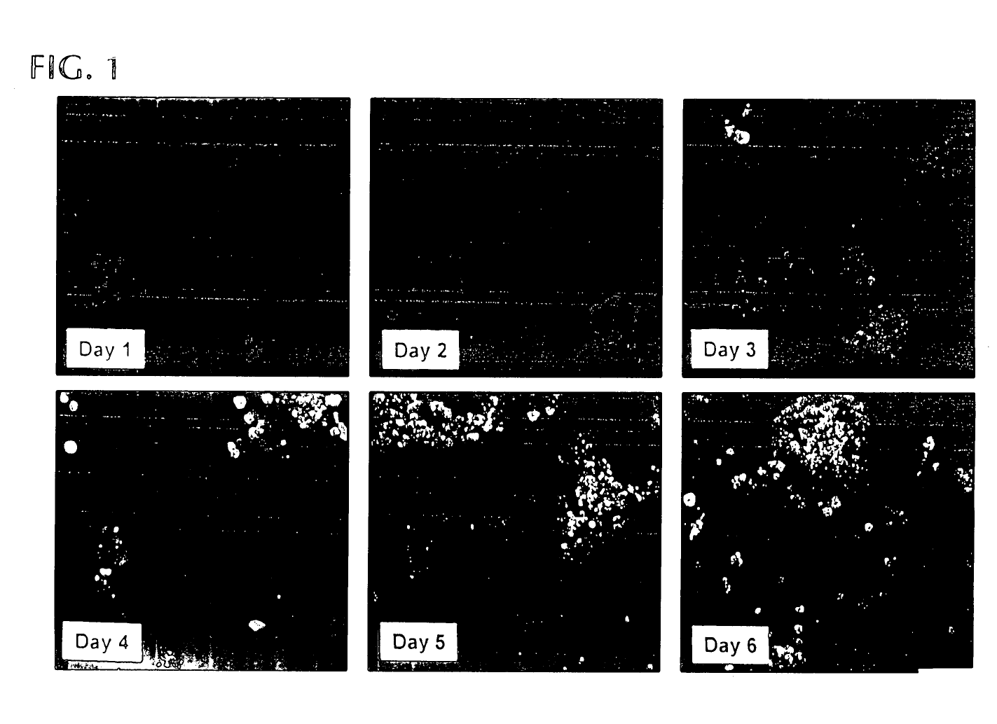

A time course of differentiating RAW cells is shown in FIG. 1. At day 1, RAW cells were mono-nucleated, small in size, and generally not proliferating. By day 2, proliferation of RAW cells began. TRAP positive staining can be detected by day 3. At day 4, cells grew in size and became increasingly TRAP positive. During days 5 and 6, large multi-nucleated TRAP positive cells were formed. These OCL at days 5 and 6 were morphologically and functionally similar to osteoclast.

Over time, RAW cells fuse and form large multinucleated cells. The general cell size and nucleation was observed in this experiment by staining in 10 ug/ml of Pi (staining the cells red). During the differentiation process, cells begin to express TRAP. Staining for TRAP was first seen at day 3 and increased over time (staining the cells yellow). Staining was visible in both large multinucleated OCL (greater than 3×mononuclear cells) and in small mononuclear cells. Staining for TRAP was achieved by providing ELF-97 as a phosphatase substrate in the presence of Tartrate.

### EXAMPLE 5

Quantification of TRAP Activity During Cell Differentiation.

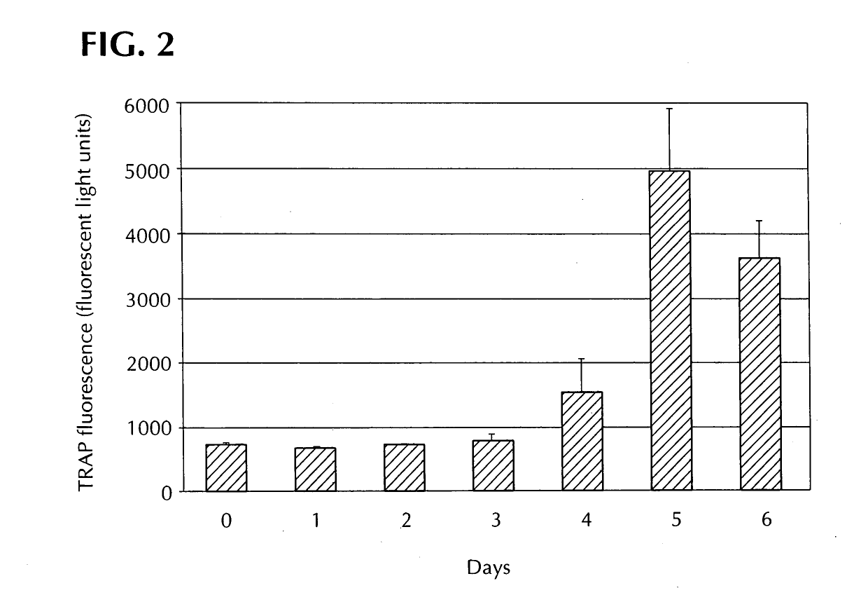

TRAP activity was assayed on a well-by-well basis utilizing total fluorescence readout. FIG. 2 is a representative graph demonstrating the increased TRAP fluorescence that was detected during the differentiation of RAW cells over time into OCL. There is little change in TRAP fluorescence detected in days 0 through day 3. An increase in TRAP fluorescence was demonstrated by day 4 which was also the largest value in % CV (25.8), suggesting that the timing of differentiation into OCL was continuous. Day 5 demonstrates a near five-fold increase in TRAP fluorescence which was attenuated by day 6.

In this experiment each time point had 12 replicates that were quantified with a spectrafluorometer. The data generated had lower variability between days 0 through day 3 with % CV (coefficient of variance) of 5.3, 4.4, 3.4, and 6.8, respectively. This low variability is to be expected as cellular fusion and TRAP activity begins at day 3. The largest variability was demonstrated during day 4 with a % CV of 25.8 while day 5 and day 6 had % CVs of 15.4 and 11.5, respectively. Since the highest CV was seen as the cells began to stain positive for TRAP it is likely that this variability is due in part to inconsistent timing of differentiation. This inconsistency in the timing of differentiation is further supported by the observations that as the cells continue through the time course of differentiation, this variability in % CV decreases.

Quantification of TRAP Activity in OCL.

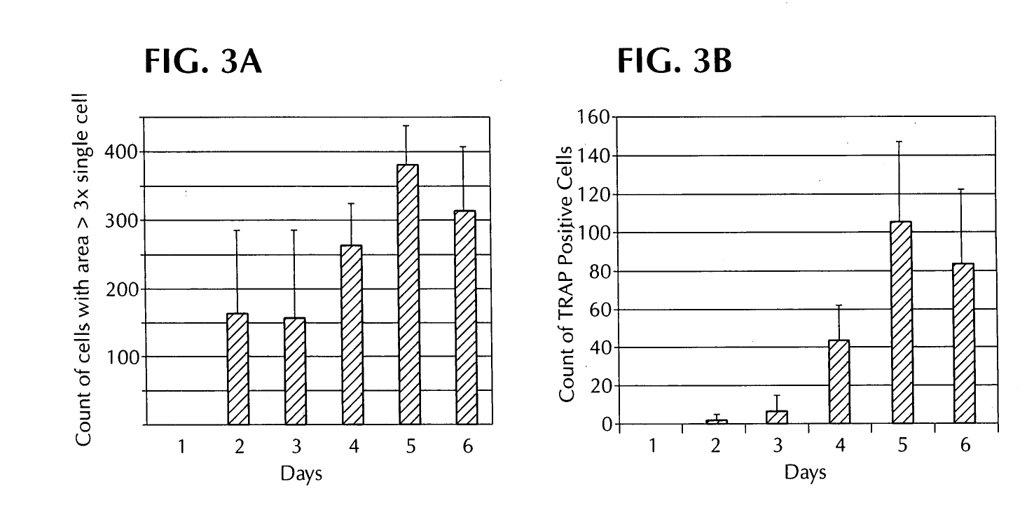

The determination of differentiation into OCL is typically based upon morphological criteria along with staining for TRAP. As seen in FIG. 1 some TRAP positive cells are small and mono-nucleated, and consequently visual scoring would not count these cells as OCL. Therefore, basing differentiation solely upon per well TRAP activity could lead to a false positive reading. To circumvent this limitation we developed an automated approach to quantify the number of OCL based upon the size of the cells in addition to TRAP activity. Due to the limitations of the software analyses, the size of the cells were used in place of number of nuclei as a selection criteria for OCL formation. The size of the cells typically correlates with cell fusion and the number of nuclei. Also while some single nucleated cells can also be large in size, these cells typically tend not to be TRAP positive. The data capture was performed using the Cellomics ArrayScan 11, which is an automated fluorescent microscope and plate handler. The data analyses were performed by using the Cellomic's “Nuclear Translocation” imaging analysis software. The results of automated counting of size selected cells are shown in FIG. 3A. Cells were counted if they had a total area that was greater than 3×that of a single mono-nucleated cell as measured at day 1. Maximum cell size was attained at day 5 and slightly attenuated by day 6. Cells counted had a total area that was greater than 3×that of a single mono-nucleated cell as measured with a 10× objective at day 1 (which were comparable in size to a non-differentiated cell). However, this criterion by itself (3×cell size) does not filter out large single nuclei cells nor does it filter out small single nuclei cells that were clustered but not expressing TRAP. Therefore, on top of the size criteria, we applied a TRAP staining intensity filter to quantify the number of OCL (FIG. 3B). Correspondingly, the maximum number of TRAP positive cells was attended by day 5 and again slightly attenuated by day 6 with progressive increases shown through out days 2, 3, and 4. The uncleaved phosphatase substrate is weakly fluorescent hence cells without TRAP activity have a background staining. By filtering out all cells with a low staining intensity we eliminated cells that were simply clustering together but lacking significant TRAP activity.

The vast majority of cells counted after selection by size and staining intensity were TRAP-positive OCL, by visual inspection. Since a sub-population of RAW cells can continue to proliferate after the initiation of differentiation, cells that are automatically counted as OCL can be small mononuclear TRAP-positive RAW cells that are clustered with proliferating RAW cells. However, these scoring events were rare and only contribute to less than 1% of all cells that were scored as OCL (data not shown).

### EXAMPLE 6

Effects of compounds that inhibit osteoclastogenesis.

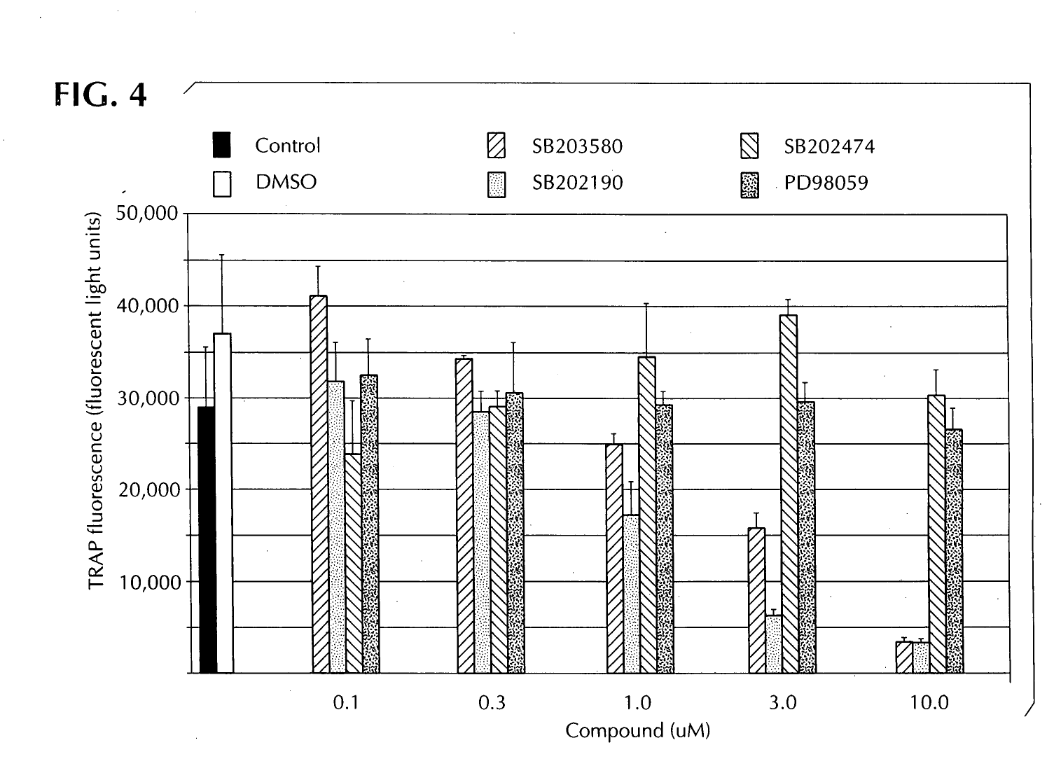

To further validate and test the utility of the assays, we employed the use of compounds known to inhibit osteoclastogenesis and observed the effects of these compounds on day six morphology and physiology. It has been demonstrated that inhibition of the p38 MAPK (mitogen-activated protein kinase) signaling pathway can block the differentiation of monocytes into osteoclasts. In particular, the p38 inhibitory compounds SB202190 and SB203580 have both shown a dose dependent inhibition of OCL formation with IC50's of 1 uM and 3 uM, respectively. Using these compounds in our assay system, both compounds demonstrated a dose dependent inhibition of TRAP activity with IC50's comparable to previously published data (FIG. 4) (see Matsumoto M, Sudo T, Saito T, Osada H, Tsujimoto M. Involvement of p38 mitogen-activated protein kinase signaling pathway in osteoclastogenesis mediated by receptor activator of NF-kB ligand (RANKL). J Biol Chem 2000; 275: 31155-31161; and KumarS, Votta BJ, Rieman DJ, Badger AM, Gowen M, Lee JC. IL-1- and TNF-induced bone resorption in mediated by p38 mitogen activated protein kinase. J Cell Physiol 2001; 187: 294-303). SB203580 and SB202190 are p38 MAPK inhibitory compounds while PD98059 is a MEK1 inhibitor and SB202474 is an inactive analog of SB202190. There was a dramatic decrease of TRAP fluorescence starting at the 1.0 uM compound amounts and that was maximal at the 10.0 uM amounts for the p38 inhibitory compounds (SB203580 and SB202190). Conversely up to 10.0 uM amounts of PD98059 and SB202474 did not significantly inhibit the detection of TRAP fluorescence. The DMSO treated cells had no detectable change from the control cells.

As expected, the MEK1 inhibitor (PD98059) and control compound (SB202474; an inactive analog of SB202190) had little effect on TRAP activity. These results demonstrate that this assay can accurately and rapidly replicate the manual scoring of compound effects and can generate dose response curves. This assay was performed in a 96 well format and could be adapted to 384 well format. Additionally, this assay only requires approximately 90 minutes to completion (from cellular fixation through analyses).

Effect of MAPK Inhibitors on OCL Numbers

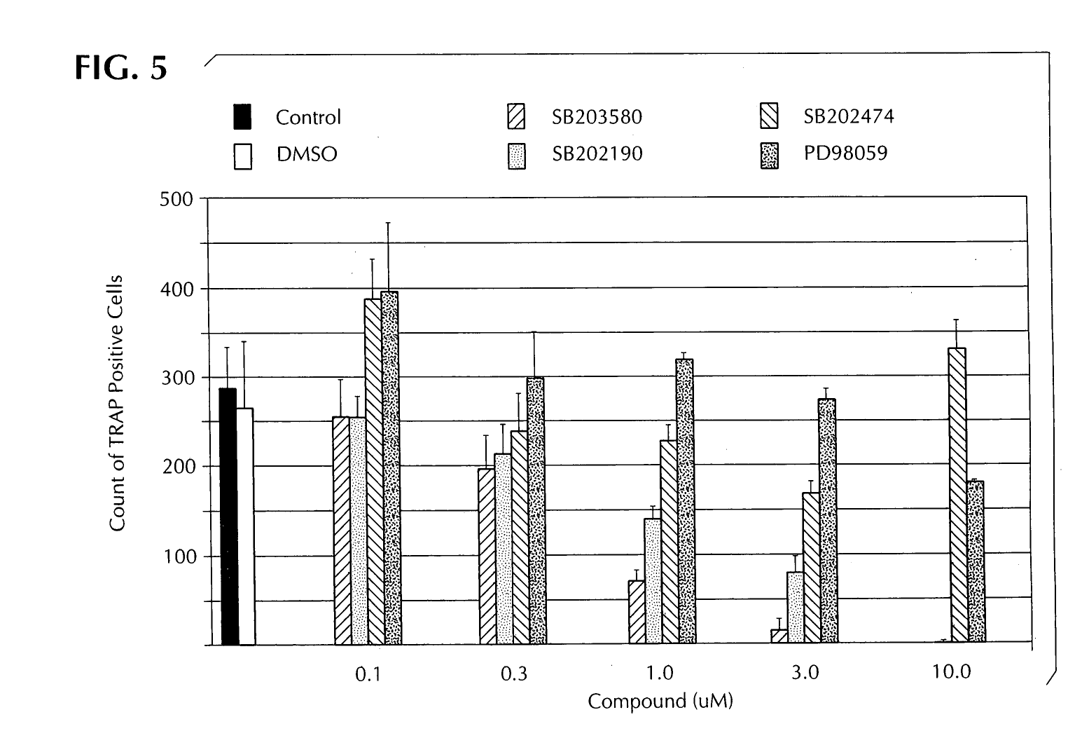

The effect of MAPK inhibitor compounds on OCL numbers was evaluated using the automated OCL counting assay (FIG. 5). SB203580 and SB202190 are p38 MAPK inhibitory compounds while PD98059 is a MEK1 inhibitor and SB202474 is an inactive analog of SB202190. The compounds were added to the cells immediately after plating and effects were quantified at day 6. There was a decrease in the quantity of TRAP positive cells counted that started at 0.3 uM compound amounts for the p38 inhibitors (SB203580 and SB202474). This decrease by p38 inhibitors was maximal and near zero amounts (for TRAP positive cell counted) at the 10.0 uM concentration. Conversely, even at the 10.0 uM amounts both PD98059 and SB202190 had no significant decrease in the number of TRAP positive cells counted. The DMSO treated cells had no detectable change from the control cells.

The p38 inhibitory compounds (SB202190 and SB203580) both demonstrated the expected profile of inhibition with IC50's between 1 uM and 3 uM (FIG. 5). As expected, the MEK1 inhibitor (PD98059) and control compound (SB202474) demonstrated little effect on the number of OCL. These results were in close agreement with well-by-well TRAP activity and recapitulated the published effects of these compounds which were scored by manual counting (see Matsumoto M, Sudo T, Saito T, Osada H, Tsujimoto M. Involvement of p38 mitogen-activated protein kinase signaling pathway in osteoclastogenesis mediated by receptor activator of NF-kB ligand (RANKL). J Biol Chem 2000; 275: 31155-31161; and Kumar S, Votta BJ, Rieman DJ, Badger AM, Gowen M, Lee JC. IL-1- and TNF-induced bone resorption in mediated by p38 mitogen activated protein kinase. J Cell Physiol 2001; 187: 294-303).

The automated OCL counting assay was performed in a 96 well format and requires approximately 3 hours to completion from cellular fixation through analyses. Due to the nature of RAW cells (proliferation of non-differentiating cells) it may not be possible to transfer this assay into 384 well format. In the current format, this assay is suitable for low to medium throughput analyses of compound- or gene-libraries for their phenotypic effects on osteoclastogenesis. However, it is likely that this assay can be adapted to evaluate other OCL systems in a 384 well high-throughput assay format (such as the utilization of CD 34(+) selected human monocytes). In addition, this “high content analysis” generates large amounts of data on changes in cellular morphology that may help track mode of action for any compound or gene.

We claim:

1. An assay for tartrate resistant acid phosphatase activity in a cell sample, comprising;
incubating a reaction mixture comprising the cell sample, a fluorescently-labeled phosphatase substrate, buffer and tartrate;
washing the reaction mixture; and
determining the amount of fluorescence of the reaction mixture.

2. The assay of claim 1 wherein the fluorescently-labeled phosphatase substrate is 2-(5′-chloro-2-phosphoryloxyphenyl)-6-chloro-4 (3H)-quinazol inone (ELF-97 phosphatase).

3. The assay of claim 1 wherein the reaction mixture is washed from about two to about five times.

4. The assay of claim 1 wherein the cells are selected from the group consisting of isolated osteoclasts, osteoclast cell lines, osteoclast like cells, and biological extracts.

5. The assay of claim 4 wherein the cells are osteoclast-like cells derived from a RAW 264.7 cell line.

6. The assay of claim 1, wherein the buffer is from about pH 4.85 to about pH 5.15.

7. The assay of claim 6, wherein the buffer is about pH 5.0.

8. The assay of claim 6 wherein the buffer is selected from the group consisting of itrate, malonate, lactate, trichloroacetate, sulfosalicylate, tartarate, phosphates, borates, acetates, piperazine-N,N′-bis(2-hydroxypropane)sulfonic acid (POPSO), N-2-hydroxythylpiperazine-N′-2-ethanesulfonic acid (HEPES), 3-N-(trishydroxymethyl)methylamino-2-hydroxypropanesulfonic acid (TAPSO), and 2-([atris-(hydroxymethyl)methyl]amino)ethanesulfonic acid (TES); or combinations thereof.

9. The assay of claim 1, wherein the concentration of tartrate is from about 6.0 mM to about 7.4 mM.

10. The assay of claim 9 wherein the concentration of tartrate is about 6.7.

11. The assay of claim 1 wherein the cells are fixed to a plate.

12. The assay of claim 1, wherein the assay is carried out on a multiwell platform.

13. A method of evaluating the degree of activity of an osteoclast modulating agent candidate comprising:
incubating a first composition comprising the candidate, a cell sample, a fluorescently-labeled phosphatase substrate, buffer and tartrate;
incubating a second composition comprising a cell sample, a fluorescently-labeled phosphatase substrate, buffer and tartrate; and
comparing the amount of fluorescence in the first composition and the second composition wherein a change in the fluorescence level indicates the degree of efficacy of the osteoclast modulating agent.

14. An assay for determining the distribution of tartrate resistant acid phosphotase in or on a cell sample comprising:
incubating a reaction mixture comprising the cell sample, a fluorescently-labeled phosphatase substrate, an acidic buffer, and tartrate;
washing the mixture; and
determining the pattern of fluorescence in the cell sample.

15. A method of screening for or monitoring the level of bone resorption in a mammalian subject, comprising:
obtaining a biological extract from a mammalian subject;
determining a level of TRAP in the sample by incubating a reaction mixture comprising the biological extract; a fluorescently-labeled phosphatase substrate; an acidic buffer; and tartrate; and determining the amount of fluorescence in the biological extract, thereby determining the level of bone resorption.

16. A kit for performing an assay for detecting tartrate resistant acid phosphatase activity, comprising:
a fluorescently-labeled phosphotase substrate;
about pH 4.85 to about pH 5.15 buffer; and
a source of tartrate.

17. The kit of claim 16 wherein the fluorescently-labeled substrate is 2-5′-chloro-2-phosphoryloxyphenyl)-6-chloro-4 (3H)-quinazolinone (ELF-97 phosphatase).
